(19) United States
(12) Patent Application Publication
(10) Pub. No.: US 2024/0299666 A1
(43) Pub. Date: Sep. 12, 2024

(54) SYRINGE CARRIERS

(71) Applicant: SHL MEDICAL AG, Zug (CH)

(72) Inventors: Chia-Hsin Su, New Taipei City (TW); Pei Yu Chao, Taoyuan City (TW); Tzu Wei Liu, Taoyuan City (TW); Slobodan Stefanov, Deerfield Beach, FL (US); Anders Boström, Ingaro (SE); Ming-Ting Yin, Taoyuan City (TW); Nurettin Ali, Boca Raton, FL (US); Justin Stewart, Deerfield Beach, FL (US)

(21) Appl. No.: 18/275,341

(22) PCT Filed: Feb. 4, 2022
(86) PCT No.: PCT/EP2022/052686

§ 371 Date: Aug. 1, 2023

Related U.S. Application Data
(60) Provisional application No. 63/149,380, filed on Feb. 15, 2021.
(60) Provisional application No. 63/153,408, filed on Feb. 25, 2021.
(60) Provisional application No. 63/158,580, filed on Mar. 9, 2021.
(60) Provisional application No. 63/162,601, filed on Mar. 18, 2021.
(60) Provisional application No. 63/178,577, filed on Apr. 23, 2021.
(60) Provisional application No. 63/287,644, filed on Dec. 9, 2021.

(30) Foreign Application Priority Data
Aug. 2, 2021 (EP) 21188997.7

(51) Int. Cl. A61M 5/24

(52) U.S. Cl. CPC A61M 5/24; A61M 2005/244

(57) ABSTRACT

The application describes syringe carriers for medicament delivery devices such as autoinjectors. One example concerns a syringe carrier for a syringe with a flange, the syringe carrier having a tubular housing extending along a longitudinal axis from a proximal end to a distal end and a syringe holder attached to the distal end of the tubular housing, wherein the syringe holder is configured to hold the flange of the syringe, and wherein the syringe holder comprises either a c-clip or a screw thread. Other associated syringe carriers, sub-assemblies, medicament delivery devices and corresponding methods are also described.

### CROSS REFERENCE TO RELATED APPLICATIONS

The present application is a U.S. National Phase application pursuant to 35 U.S.C. § 371 of International Application No. PCT/EP2022/052686 filed Feb. 4, 2022, which claims priority to U.S. Provisional Patent Application No. 63/149,380, filed Feb. 15, 2021; U.S. Provisional Patent Application No. 63/153,408, filed Feb. 25, 2021; U.S. Provisional Patent Application No. 63/158,580, filed Mar. 9, 2021; U.S. Provisional Patent Application No. 63/162,601, filed Mar. 18, 2021; U.S. Provisional Patent Application No. 63/178,577, filed Apr. 23, 2021; U.S. Provisional Patent Application No. 63/287,664 filed Dec. 9, 2021; and European Patent Application No. 21188997.7 filed Aug. 2, 2021. The entire disclosure contents of these applications are herewith incorporated by reference into the present application.

### TECHNICAL FIELD

Syringe carriers for medicament delivery devices such as autoinjectors.

### BACKGROUND

Medicament delivery devices such as autoinjectors often have a syringe carrier to support a syringe (typically a glass syringe) within the medicament delivery device. Although existing syringe carriers can be effective at supporting the syringe, the inventors have appreciated that further improvements can be made.

### SUMMARY

The present disclosure concerns a number of different concepts for supporting a syringe in a medicament delivery device as described below.

The present disclosure is defined by the appended claims, to which reference should now be made.

An aspect concerns a syringe carrier for a syringe with a flange, the syringe carrier comprising a tubular housing extending along a longitudinal axis from a proximal end to a distal end and a syringe holder attached to the distal end of the tubular housing, wherein the syringe holder is configured to hold the flange of the syringe, and wherein the syringe holder comprises either a c-clip or a screw thread.

Optionally, the syringe holder comprises a c-clip, and the c-clip extends more than half of the way around the circumference relative to the longitudinal axis, and preferably more than 270 degrees around the circumference relative to the longitudinal axis.

Optionally, the syringe holder comprises a c-clip, and the c-clip is flexible to allow the diameter of the c-clip to expand to allow the flange of the syringe to pass the c-clip during medicament delivery device assembly.

Optionally, the syringe holder comprises a c-clip, and the c-clip comprises two inwardly extending ribs extending around the circumference of the c-clip, wherein the two inwardly extending ribs are spaced apart from one another in the direction of the longitudinal axisso that the flange of the syringe can fit between the two inwardly extending ribs.

Optionally, the syringe holder comprises a screw thread, and wherein the screw thread extends towards the longitudinal axis from an inside surface of the syringe holder. Optionally, the syringe holder is tubular.

Another aspect concerns a sub-assembly for a medicament delivery device, the sub-assembly comprising a syringe carrier as described above and a syringe with a flange, wherein the syringe is inside the syringe carrier and the flange is inside the syringe holder.

Another aspect concerns a medicament delivery device comprising the syringe carrier described above or the sub-assembly described above, wherein the medicament delivery device is optionally an autoinjector.

Another aspect concerns a method of inserting a syringe into a syringe carrier, the method comprising the steps of: providing a syringe carrier according to any of claims 1 to 6 and providing a syringe with a flange; and inserting the syringe into the syringe carrier in the direction of the longitudinal axis.

Optionally, the syringe is inserted into the distal end of the syringe carrier and moved in the proximal direction relative to the syringe carrier until the flange of the syringe engages the syringe holder.

Optionally, the syringe holder comprises a screw thread and the syringe is rotated relative to the syringe carrier during insertion of the syringe into the syringe carrier so as to engage the flange of the syringe with the screw thread.

Another aspect concerns a medicament delivery device extending along a longitudinal axis in an axial direction from a distal end to a proximal end, the medicament delivery device comprising: a housing; a medicament delivery member guard attached to the housing and axially moveable relative to the housing from a proximal position to a distal position, the medicament delivery member guard comprising a surface that faces towards the longitudinal axis and extends in the axial direction; a tubular syringe carrier attached to the housing, wherein the syringe carrier comprises a flexible arm at a proximal end of the syringe carrier, the flexible arm comprising a protrusion extending towards the longitudinal axis; and a syringe inside the syringe carrier, wherein, when the medicament delivery member guard is in the distal position, the surface of the medicament delivery member guard is adjacent to the flexible arm of the syringe carrier so as to restrict movement of the protrusion of the flexible arm away from the longitudinal axis, and when the medicament delivery member guard is in the proximal position, movement of the protrusion of the flexible arm away from the longitudinal axis is not restricted by the surface of the medicament delivery member guard, so that the protrusion of the flexible arm is free to move away from the longitudinal axis. This can allow the protrusion on the flexible arm to support a shoulder of the syringe carrier when the medicament delivery member guard is in the distal position.

Optionally, the flexible arm of the syringe carrier is attached to the rest of the syringe carrier at a distal end of the arm.

Optionally, the flexible arm of the syringe carrier comprises an outwardly extending protrusion, and wherein, when the medicament delivery member guard is in the distal position, the surface of the medicament delivery member guard is adjacent to the outwardly extending protrusion.

Optionally, there is a gap between the protrusion of the flexible arm and a shoulder of the syringe carrier when the medicament delivery member guard is in the proximal position and there is no gap between the protrusion of the flexible arm and the shoulder of the syringe carrier when the medicament delivery member guard is in the distal position.

Optionally, the medicament delivery device comprises a damper at a distal end of the syringe carrier. This can support and cushion a flange of the syringe. Optionally, the damper is an integral part of the syringe carrier. Optionally, the damper is a separate component at the distal end of the syringe carrier.

Optionally, the damper is a compression spring. Optionally, the damper is a flexible ring or a flexible protrusion. Optionally, the damper is made of a flexible material such as a thermoplastic elastomer.

Optionally, the medicament delivery member guard comprises a ring at a proximal end and two arms extending from a distal end of the ring.

Optionally, the surface of the medicament delivery member guard is an inner surface of one of the arms of the medicament delivery member guard. Optionally, the syringe carrier is an integral part of the housing.

Optionally, the medicament delivery device is an autoinjector.

Another aspect concerns a damping ring for a medicament delivery device, the damping ring being made of a flexible material, the damping ring comprising two arms, wherein the arms are configured to engage a component of said medicament delivery device to hold the damping ring in place relative to said component of said medicament delivery device.

Another aspect concerns a housing for a medicament delivery device, the housing extending along a longitudinal axis from a proximal end to a distal end, the housing comprising a tubular body and one or more flexible ribs extending inwards towards the longitudinal axis from the tubular body, wherein the flexible ribs extend from the tubular body at an angle relative to a radial direction relative to the axis so that, when a syringe is placed inside the tubular body, the one or more flexible ribs flex away from the axis and hold the syringe. Optionally, the angle is between 10 and 90 degrees, preferably between 20 and 60 degrees, most preferably between 30 and 45 degrees. Optionally, four flexible ribs are provided.

Another aspect concerns a medicament delivery device comprising the damper ring described above or the housing described above. Optionally, the medicament delivery device is an autoinjector.

Another aspect concerns a method of assembling a medicament delivery device, comprising inserting a syringe into a housing so that the syringe causes a rib inside a tubular body of the housing to flex away from a longitudinal axis of the housing, thereby supporting the syringe within the housing.

### BRIEF DESCRIPTION OF THE DRAWINGS

Embodiments of the present disclosure will now be described by way of example only and with reference to the accompanying drawings as listed below.

FIGS. 1, 2 and 3 show cross-section views of a syringe carrier and a syringe during and after syringe insertion.

FIG. 4 shows an end view of the syringe carrier of FIG. 1.

FIG. 5 shows a perspective view of the syringe carrier of FIG. 1.

FIGS. 6, 7 and 8 show cross-section views of a syringe carrier and a syringe during and after syringe insertion.

FIG. 9 shows an end view of the syringe carrier of FIG. 6.

FIG. 10 shows a perspective view of the syringe carrier of FIG. 6.

FIG. 11 shows a perspective view of a medicament delivery device with some components shown as partially see-through.

FIG. 12 shows a perspective view of the medicament delivery device of FIG. 11.

FIG. 13 shows a perspective cross-section view of part of the medicament delivery device of FIG. 11.

FIG. 14 shows a cross-section view of part of the medicament delivery device of FIG. 11.

FIG. 15 shows a cross-section view of part of the medicament delivery device of FIG. 11.

FIGS. 16, 17 and 18 show different views of a syringe carrier.

FIGS. 19, 20 and 21 show different views of a syringe carrier.

FIGS. 22, 23 and 24 show different views of a syringe carrier.

FIG. 25 shows a perspective view of a syringe carrier.

FIG. 26 shows a perspective view of a syringe carrier.

FIG. 27 shows a perspective view of a syringe carrier.

FIG. 28 shows a cross-section view of a housing.

FIG. 29 shows a cross-section view of a syringe carrier.

FIG. 30 shows a cross-section view of a syringe.

FIG. 31 shows a cross-section view of the syringe carrier of FIG. 29 in the housing of FIG. 28.

FIG. 32 shows a cross-section view of the syringe of FIG. 30 in the syringe carrier of FIG. 29 in the housing of FIG. 28.

FIG. 33 shows a perspective view of a syringe carrier.

FIG. 34 shows a cross-section view of a syringe in the syringe carrier of FIG. 33.

FIG. 35 shows an end view of the sub-assembly shown in FIG. 34.

FIG. 36 shows a cross-section view of a housing.

FIG. 37 shows a cross-section view of a syringe.

FIG. 38 shows a cross-section view of the syringe of FIG. 37 in the housing of FIG. 36.

FIG. 39 shows a perspective view of part of an alternative housing.

FIG. 40 shows a cross-section view of part of a medicament delivery device.

FIG. 41 shows a cross-section view of the housing of the medicament delivery device of FIG. 40.

FIG. 42 shows a cross-section perspective view of part of the medicament delivery device of FIG. 40.

FIG. 43 shows a cross-section view of a housing.

FIG. 44 shows a cross-section view of part of a powerpack.

FIG. 45 shows a cross-section view of a syringe.

FIG. 46 shows a cross-section view of the syringe of FIG. 45 attached to the powerpack of FIG. 44.

FIG. 47 shows a cross-section view of the syringe of FIG. 45 attached to the powerpack of FIG. 44 and the housing of FIG. 43.

FIG. 48 shows a perspective view of an example autoinjector.

FIG. 49 shows a perspective view of an example syringe.

FIG. 50 shows a cross-section view of the example syringe of FIG. 49.

FIGS. 51 and 52 show perspective views of a damping ring.

FIG. 53 shows a perspective view of another damping ring.

FIG. 54 shows a cross-section side view of a housing with the damping ring of FIG. 53 inserted.

FIG. 55 shows a cross-section view through part of the housing and damping ring of FIG. 54.

FIG. 56 shows a perspective view of part of the housing of FIG. 54.

FIG. 57 shows a perspective cross-section view of part of a housing with a damping portion.

FIG. 58 shows an end-on view of a housing with flexible ribs.

FIG. 59 shows a cross-section side view of part of the housing of FIG. 58 with a syringe.

FIG. 60 shows a perspective cross-section view of part of the housing of FIG. 58.

FIG. 61 shows a cross-section side view of the housing of FIG. 58.

FIG. 62 shows a partially see-through end-on view of a medicament delivery device comprising the housing of FIG. 58.

### DETAILED DESCRIPTION

Embodiments of the present disclosure will now be described by way of example only and with reference to the accompanying drawings. The present disclosure is defined by the appended claims, to which reference should now be made.

In general, the present disclosure describes various syringe carriers for medicament delivery devices such as autoinjectors. Generally, these syringe carriers are for use in devices in which the syringe is inserted into the device (for example into a housing of the device) in the axial direction during assembly. The focus in this application is primarily on syringe carriers that support a syringe at the flange.

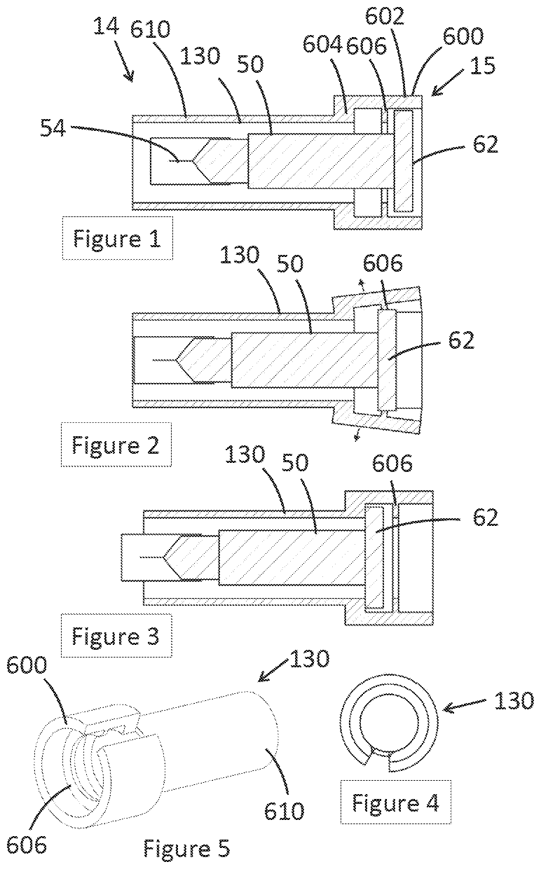

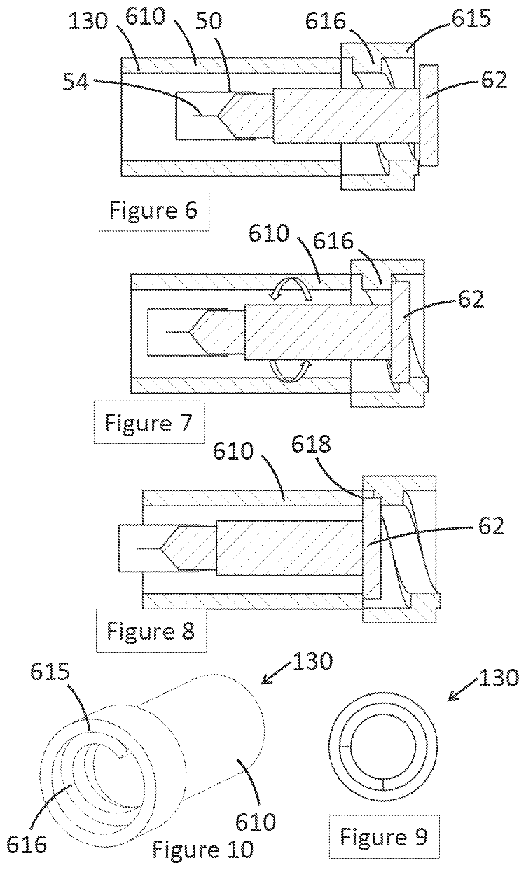

In one example, depicted in FIGS. 1 to 5, the distal end of the syringe carrier 130 comprises a syringe holder, in this case a c-clip 600. FIG. 1 shows the syringe 50 being inserted into the syringe carrier in the axial direction. FIG. 2 shows the flange 62 of the syringe pushing into the c-clip 600. FIG. 3 shows the final position of the syringe 50 relative to the syringe carrier 130. FIG. 4 shows a view of the syringe carrier (particularly the c-clip 600) in the axial direction 13. FIG. 5 shows the syringe carrier 130. This is an example of a design in which axial movement of the syringe in either direction (distal or proximal) can be restricted (either minimised or stopped entirely) by the syringe carrier. To provide this restriction, the c-clip 600 comprises a tubular section 602 with a distal rib 606 and a proximal rib 604, with the proximal rib 604 closer to the proximal end of the syringe carrier than the distal rib 606. The proximal rib protrudes towards the longitudinal axis from the inner surface of the tubular section 602 (that is, it is a radially inwardly extending rib). Similarly, the distal rib protrudes towards the longitudinal axis from the inner surface of the tubular section 602 (that is, it is a radially inwardly extending rib). The proximal and distal ribs are spaced apart from one another in the axial direction so that the flange 62 can fit between the ribs. Optionally, padding (not shown)(such as a thermoplastic elastomer (TPE)) is provided between the ribs to cushion the flange. The c-clip is flexible to allow the flange of the syringe to pass the distal rib during assembly. Preferably, the c-clip is only flexible enough to allow the flange of the syringe to pass the distal rib but not to pass the proximal rib. The particular shape of the c-clip in the example shown in the figures is not essential. For example, the shape of the ribs 604, 606 as shown in the figures is not essential, and one or more of the ribs could comprise one or more protrusions rather than a rib—this would still be sufficient to hold the flange in place. Another alternative would be to provide a recess into which the flange fits, rather than the two ribs. In general, the c-clip is configured to hold the flange of the syringe carrier.

In a method of assembly of a medicament delivery device sub-assembly comprising a syringe carrier and a syringe, the proximal end of the syringe is inserted in the axial direction into the distal end of the syringe carrier. As the syringe moves in the proximal direction relative to the syringe carrier, the c-clip 600 then expands to allow the flange of the syringe to pass the distal rib 606 of the c-clip 600 (FIG. 2). Once the flange has passed the distal rib 606, the c-clip 600 relaxes back into place, thereby restricting movement of the syringe in the distal direction relative to the syringe carrier. The same movement would also occur with a differently shaped c-clip, with the c-clip (or part of the c-clip) flexing outwards to let the syringe flange enter the c-clip and thereby to engage the c-clip.

When the syringe carrier is placed in its final position in a medicament delivery device, there is optionally a feature of another component of the medicament delivery device, such as a protrusion of a housing, which is adjacent to the syringe holder so that the syringe holder cannot flex outwards and release the syringe when in a completed medicament delivery device.

Typically, the c-clip extends more than half of the way around the circumference of the syringe holder relative to the longitudinal axis. Preferably, the c-clip extends more than 270 degrees (three-quarters of the way) around the circumference of the syringe holder relative to the longitudinal axis. Preferably, the c-clip extends between 270 and 350 degrees around the circumference of the syringe holder relative to the longitudinal axis, or between 300 and 350 degrees around the circumference of the syringe holder relative to the longitudinal axis or between 300 and 330 degrees around the circumference of the syringe holder relative to the longitudinal axis. The particular angle may depend on the design of the syringe carrier and of the syringe, but it can be beneficial for the c-clip to extend more than 270 degrees around the circumference of the syringe holder relative to the longitudinal axis to help support the syringe evenly.

In this example, the distal end of the tubular portion 610 (tubular housing) of the syringe carrier is attached to the proximal rib, although the distal end of the tubular portion 610 of the syringe carrier could alternatively be attached to the tubular portion 602 of the c-clip.

In another example (FIGS. 6 to 10), another design in which axial movement of the syringe in either direction (distal or proximal) can be restricted by the syringe carrier is provided. In this example, the distal end of the syringe carrier 130 comprises a syringe holder 615, in this case comprising a helically extending rib 616 (screw thread). During assembly (e.g. FIGS. 6 and 7) the syringe is rotated relative to the syringe carrier to screw the flange through the screw thread. FIG. 8 shows the syringe 50 inserted in the syringe carrier 130. FIG. 9 shows a view in the axial direction 13 of the syringe carrier. FIG. 10 shows the syringe carrier 130.

The syringe carrier 130 comprises a tubular portion 610 and a syringe holder 615 attached to the distal end of the tubular portion 610. The syringe holder is tubular, and comprises a helically extending rib 616 that extends towards the longitudinal axis from the inside surface of the tubular portion 610 of the syringe holder. When in the final position inside the syringe carrier, the flange of the syringe is between the distal end 618 of the tubular portion 610 of the syringe carrier 130 and the helically extending rib 616. Alternatively, instead of using the distal end 618 of the tubular portion 610, the syringe holder could instead comprise a feature to stop the proximal movement of the flange, such as a proximal rib as described in the examples above.

In a method of assembly of a medicament delivery device sub-assembly comprising a syringe carrier and a syringe, the proximal end of the syringe is inserted in the axial direction into the distal end of the syringe carrier. As the syringe moves in the proximal direction relative to the syringe carrier, the syringe reaches a point (FIG. 6) where the syringe cannot move any further in the proximal direction relative to the syringe carrier without also rotating. At this point, the syringe is rotated relative to the syringe carrier (FIG. 7), with the syringe reaching a final position between the distal end 618 of the tubular portion 610 of the syringe carrier 130 and the helically extending rib 616 (screw thread).

Optionally, the outer diameter of the syringe can be the same as (or slightly larger than) the inner diameter of the tubular body (tubular portion 610) of the syringe carrier; this can help support the syringe. Optionally, the syringe holder comprises a snap fit (for example on the helically extending rib) to stop the syringe from moving back in the distal direction relative to the syringe carrier after the syringe has been inserted into the syringe carrier.

In terms of assembly of these examples, the syringe can be inserted into the syringe carrier, with the syringe and syringe carrier subsequently inserted into a housing. Alternatively, the syringe carrier can be inserted into a housing, and then the syringe can be inserted into the syringe carrier and the housing.

In another example, a damping portion as described below is provided, and the syringe carrier also has arms at the proximal end. After assembly, the syringe carrier is supported by its flange. During medicament delivery, the syringe carrier is instead (or additionally) supported at the shoulder.

During assembly, flexible arms of the syringe carrier can flex outwards, for example to allow an RNS of the syringe to pass. After assembly, the syringe carrier is not supported by the arms. There is typically a small gap between the protrusions on the arms and the syringe shoulder, though this is optional. Regardless, if the syringe moves in the proximal direction relative to the syringe carrier, the damping portion at the distal end of the syringe carrier supports the syringe-if the shoulder of the syringe reaches the arms, it would simply push the arms out away from the axis.

During cap removal, pulling the cap in the proximal direction relative to the housing could pull the syringe in the proximal direction as well. This movement of the syringe would be limited by the damping portion, which would then pull the syringe back in the distal direction once the cap is removed.

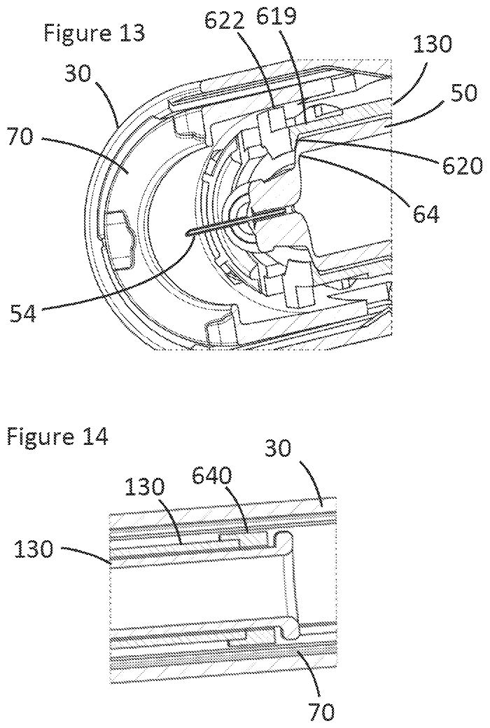

When the needle guard 70 is pushed in the distal direction during medicament delivery, the needle guard 70 is then in the way of the arms and stops the arms from moving outwards. The arms can then help with supporting the syringe during medicament delivery. This can be beneficial as it can help reduce the forces on the flange of the syringe during medicament delivery. This can be seen in FIG. 13 in particular, in which the needle guard 70 (or more generally medicament delivery member guard) is in a distal position relative to the housing (and therefore also relative to the syringe carrier).

When the needle guard is in the distal position (which in this case means that the proximal end of the housing is at the same position in the longitudinal direction as the proximal end of the needle guard, though the needle guard could also extend beyond the proximal end of the housing), the needle guard stops the outwardly extending protrusion 622, and therefore also the rest of the flexible arm 619 of the syringe carrier 130, from moving away from the longitudinal axis, even when a proximally directed force is applied to the inwardly extending protrusion 620 of the flexible arm, for example by a plunger rod pushing a stopper of the syringe during medicament delivery. As a result, when the needle guard is in the distal position (which it would be during medicament delivery), the needle guard 70 stops the flexible arm 619 from flexing, thereby providing support on the shoulder of the syringe during medicament delivery.

In one example, the outwardly extending protrusion 622 is restricted from moving away from the longitudinal axis by an inner wall of the needle guard. The needle guard can comprise a base and a plurality of arms (two arms in this example). The base can be tubular (for example ring-shaped). The arms extend from the distal end of the base. The inner wall of the needle guard can be on the base of the needle guard or on an arm of the needle guard. In this particular example, it is envisioned that protrusions 624 at the distal end of the arms engage a rotator to trigger medicament delivery when the needle guard is moved from the proximal position to the distal position relative to the housing, though other methods to trigger medicament delivery could also be used, for example with a button on the medicament delivery device.

When the needle guard is in the proximal position, the needle guard does not limit (or limits to a lesser extent) movement of the outwardly extending protrusion 622 away from the longitudinal axis. This could be by provision of a recess or cut-out in the inner surface of the needle guard 70 (either in the base or in an arm in this example), or by simply designing the needle guard so that the needle guard is not aligned with the outwardly extending protrusion 622 in the radial direction when the needle guard is in the proximal position.

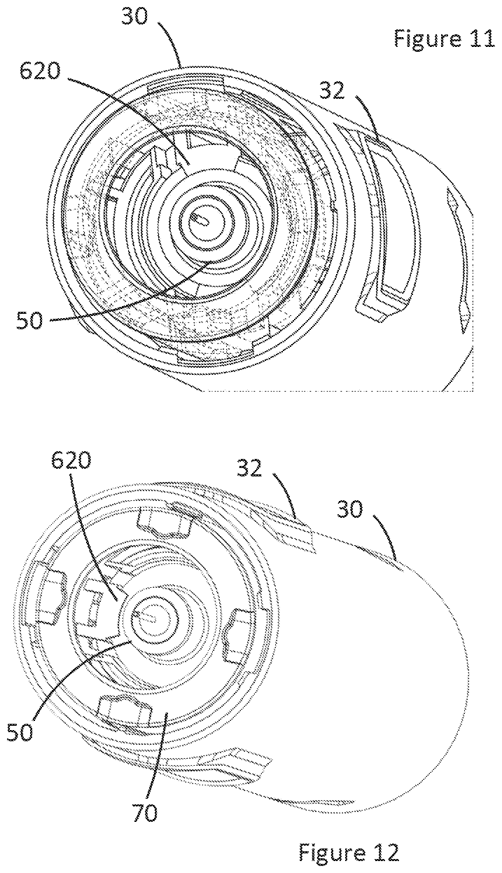

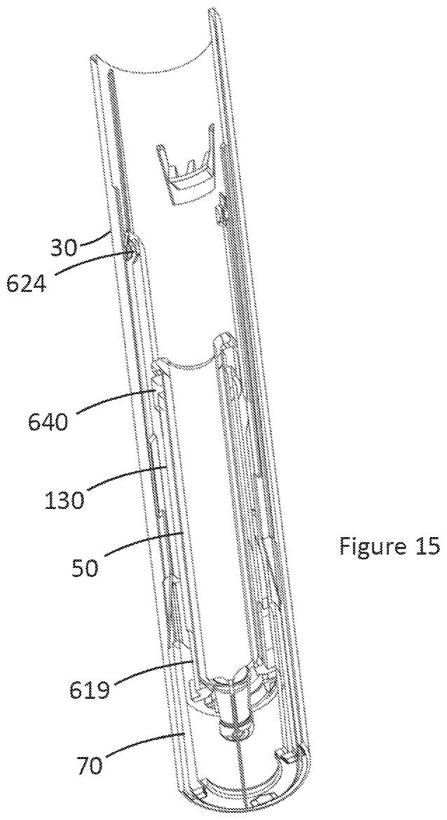

An example of this is shown in FIGS. 11 to 15. In this particular example, the damping portion is a flexible ring 640 (for example a flexible thermoplastic elastomer (TPE) ring), although other damping portions as described below could also be used. In particular, FIG. 14 shows an example damping portion, and FIGS. 11 to 13 and 15 show the position of the arms and the needle guard once the needle guard is pushed in the distal direction for medicament delivery.

The particular example shown in FIGS. 11 to 15 will now be described in more detail. A syringe carrier 130 (which in this example is an integral part of a housing 30, though it could alternatively be a separate component) comprises a flexible arm (619)(specifically two flexible arms). The syringe carrier is tubular. The distal end of the arm is attached to the rest of the syringe carrier, and the proximal end of the arm is free to move relative to the syringe carrier. The arm comprises an inwardly extending protrusion 620 and an outwardly extending protrusion 622. The inwardly extending protrusion 620 and the outwardly extending protrusion 622 are attached to the rest of the arm 619 at the proximal end of the arm (one or both of the protrusions could also be elsewhere on the arm, though they would typically be nearer the proximal end of the arm than the distal end of the arm—this can help allow the protrusion to move enough when the arm flexes so that the syringe can pass the protrusion during assembly).

A syringe 50 is arranged inside the syringe carrier 130. The inwardly extending protrusion 620 is configured to support a shoulder 64 of the syringe 50. In this example, a distally facing surface of the inwardly extending protrusion 620 is angled so that abuts the shoulder 64, though the shape could be varied depending on the particular syringe (especially the particular shape of the RNS, the shoulder and/or the gap between the RNS and shoulder).

Alternatively, the arms could be in a flexed outwards position in the final assembled device (in such a configuration, the arms could already be out of the way and therefore wouldn't be pushed out of the way when the syringe is inserted into the syringe carrier). When the needle guard is moved in the distal direction relative to the syringe carrier to carry out medicament delivery, the needle guard then pushes the arms towards the longitudinal axis, thereby pushing them towards the syringe and into a position where they support the syringe (specifically by supporting the shoulder of the syringe).

The example shown includes the arms and the damping ring provided as an integral part of another component, in this case a housing of a medicament delivery device-alternatively the syringe carrier could be a separate component arranged inside the housing.

Examples which provide a damping portion (damper) which provides a cushioned support on the distal end of the syringe carrier will now be described. This can help reduce the forces imparted to the syringe by the syringe carrier, for example during medicament delivery or when a medicament delivery device containing the syringe and the syringe carrier is dropped.

These solutions can also provide a simpler assembly process by removing the need to have the syringe carrier in an intermediate position during an intermediate stage of the assembly process—the syringe carrier can be directly placed in its final position within the medicament delivery device (typically within the housing of the medicament delivery device). Optionally, the syringe carrier could be an integral part of a housing of a medicament delivery device.

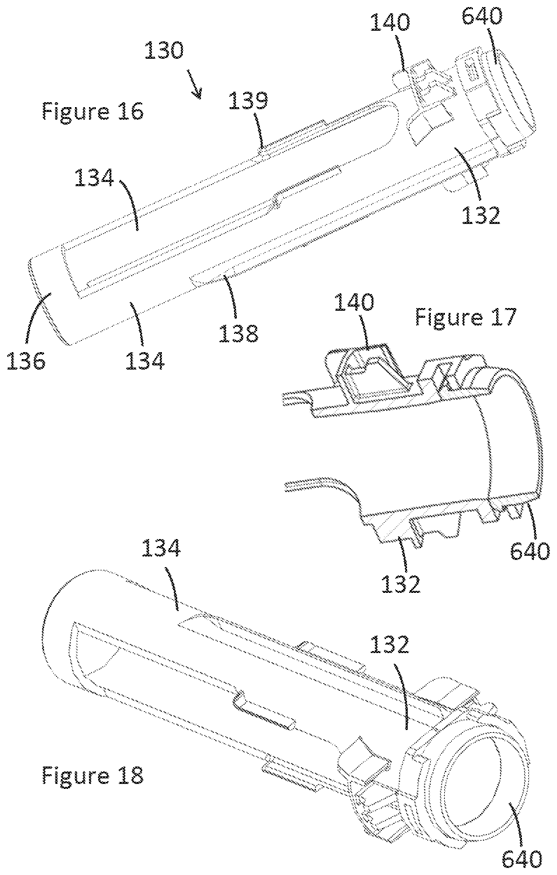

Various syringe carriers already described herein could be used with a damping portion. Further specific examples will now also be given. A first example is shown in FIGS. 16 to 18. The syringe carrier 130 in this example comprises a base 132, two arms 134 extending in the proximal direction from the base 132, a ring 136 attached to the proximal end of the arms, and various optional protrusions 138, 139, 140. However, the particular shape of the syringe carrier itself is not the focus in this case, and the focus is on the distal end of the syringe carrier, which comprises a damping portion. In this example the damping portion is a ring 640, which in this example is attached to the base 132 by a snap fit (in this case, the snap fit comprises a protrusion on the damping ring 640 and a cut-out on the base 132 as can be seen in FIG. 17 in particular, though the protrusion could be on the base and the cut-out on the damping ring, or another snap-fit structure could be used). Instead of a snap-fit, other attachment options could be used, such as overmoulding or glue.

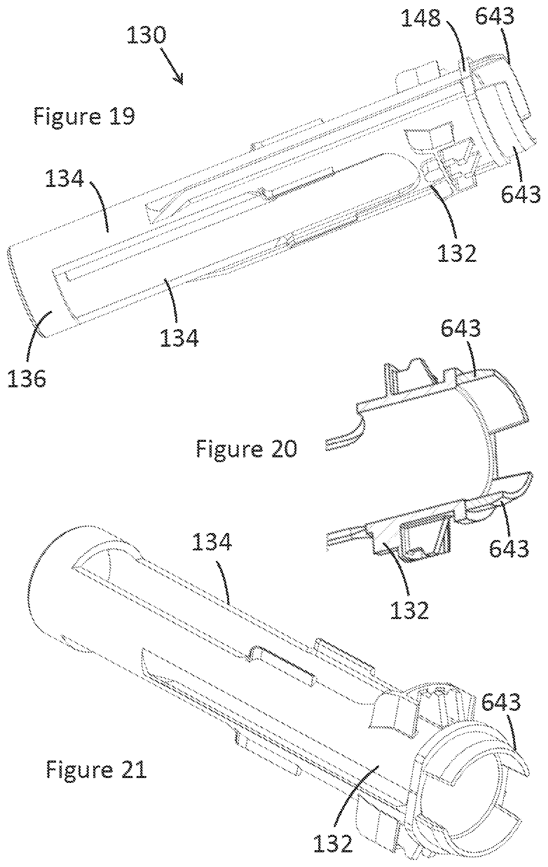

A second example is shown in FIGS. 19 to 21, in which the damping portion is two protrusions 643 extending from the distal end of the housing; in this example, it is envisioned that the protrusions would be attached by overmoulding (although other attachment options could also be used, such as glue or a snap-fit). Instead of two protrusions, one, three or more protrusions could be provided. An optional distal flange 148 can be provided; this can be beneficial as it can increase the surface area available for attaching the overmoulded part. In this example, the overmoulding is provided onto a planar surface (of the flange 148); this surface could optionally be roughened (for example by adding one or more protrusions to the surface). This can strengthen the adherence of the overmoulded part to the rest of the syringe carrier.

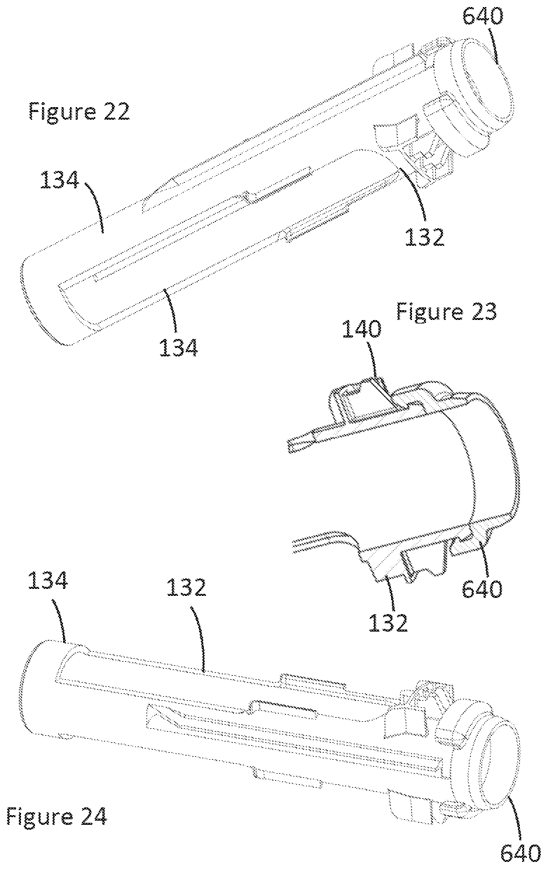

A third example is shown in FIGS. 22 to 24. In this example the damping portion is a ring 640, which in this example is again attached to the distal end of the housing by a snap fit, although in contrast to FIGS. 16 to 18, the snap fit is arranged on the outside of the base rather than on the inside of the base, and is created by a hook on the base and a corresponding hook on the ring 640, although other attachment methods such as overmoulding or glue could alternatively be used with this or similar designs.

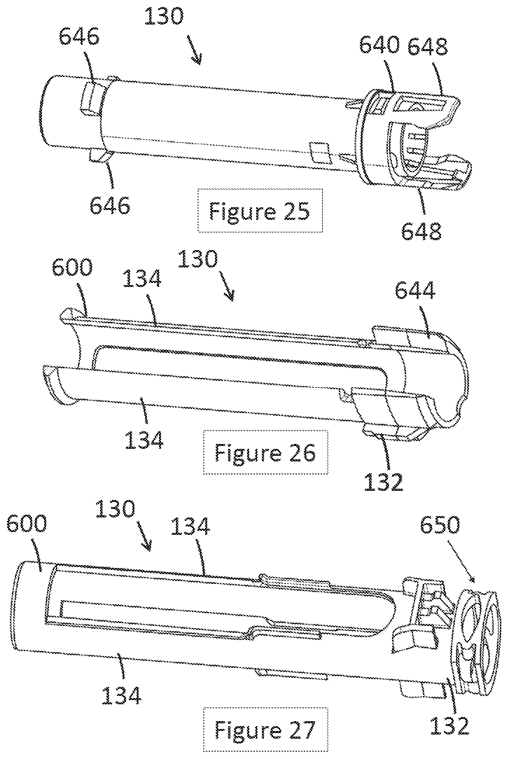

Two further examples are shown in FIG. 25 (primarily envisioned as a snap fit, though other attachment methods such as overmoulding or glue could also be used with this or similar designs) and FIG. 26 (primarily envisioned for overmoulding, though other attachment methods such as overmoulding or glue could also be used with this or similar designs).

The ring 640 in the example in FIG. 25 also comprises distally extending arms 648, which can help hold the flange of the syringe in place. The syringe carrier in FIG. 25 is tubular, and therefore does not have arms 134 (although arms 134 could optionally be included in these examples).

The syringe carrier in FIG. 26 is also tubular in shape and has arms 134 and a c-clip 600. The base 132 does not extend all the way around the axis in the circumferential direction, and the syringe carrier does not extend all the way around the axis in the circumferential direction. The damping portion 644 is also c-shaped and does not extend all the way around the axis in the circumferential direction, although a ring or protrusions as in the previous examples could alternatively be provided.

In general, in designs with a damping portion at the end such as those described above, the damping portion could be attached to the rest of the syringe carrier by various methods, including overmoulding, gluing and/or a snap fit.

Another approach for the damping portion is shown in FIG. 27, in which the damping portion is a spring 650 (specifically a compression spring). In this case the spring is moulded as an integral part of the syringe carrier, though the spring could also be a separate component.

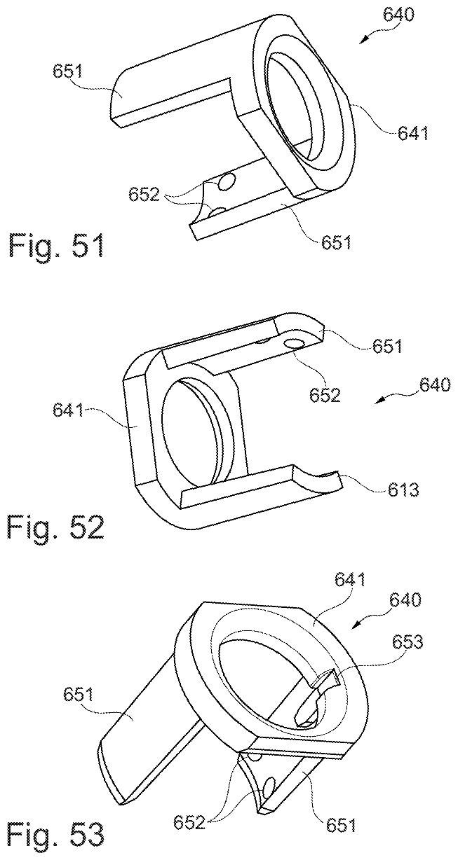

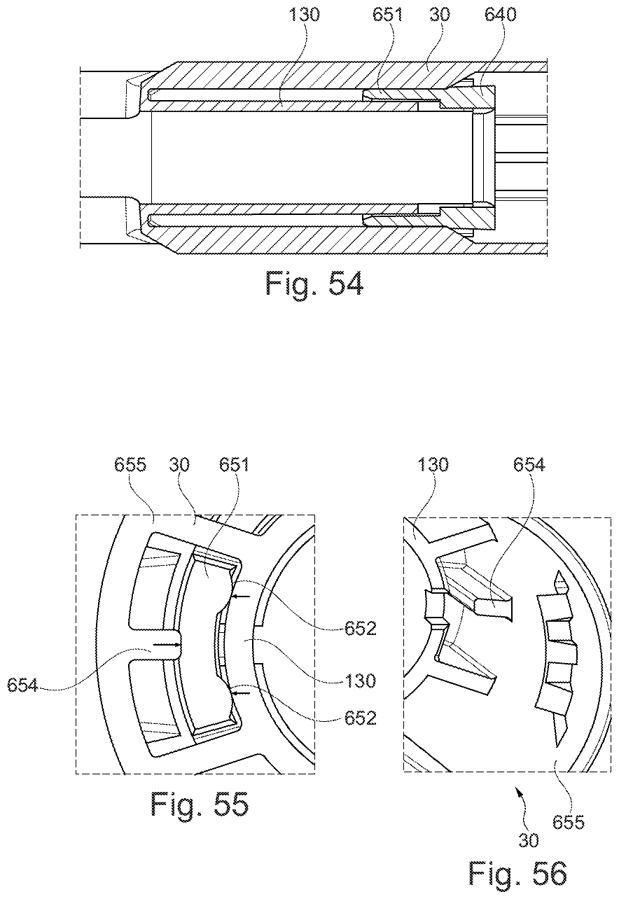

FIGS. 51 to 56 show examples of another damping portion, in this case a damping ring. As shown in FIGS. 51 and 52, a damping ring 640 comprises a base 641 and two proximally extending arms 651. Each proximally extending arm comprises two inwardly extending protrusions 652 (i.e. protrusions extending from the arm towards the axis). In a second similar example in FIG. 53, a notch 653 is also provided; the notch is in the base on an inner side of the base (i.e. the side of the base closest to the axis). The notch is optional and can help align the ring relative to other components, particularly during assembly. FIG. 54 shows the ring 640 in place in a housing 30. In this case, the housing includes a syringe carrier 130 as an integral part of the housing 30, though the syringe carrier could also be a separate component. FIG. 55 shows how the proximally extending arms 651 interact with the housing, with the protrusions 652 abutting the syringe carrier 130 (i.e. an inner part of the housing) and with a rib 654 (or alternatively a protrusion) that extends inwards (i.e. towards the axis) from an outer part 655 of the housing abutting the other side of the proximally extending arm 651 from the protrusions 652. With appropriately shaped components and the proximally extending arm 651 being made of a flexible material, the proximally extending arm 651 is flexed slightly by this arrangement, which can hold the ring 640 firmly in place relative to the housing. FIG. 56 shows another view of the housing, showing the position of the rib 654 in particular. Instead of a rib on the body and two inwardly extending protrusions on the housing, the protrusions could be removed and replaced by further ribs or protrusions on the housing. Similarly, the rib could be removed and replaced by a further protrusion on the proximally extending arm.

The base 641 is shown as a circular ring, but does not have to be circular and could be another shape, depending for example on the shape of other medicament delivery device components. Two proximally extending arms 651 are shown, although one, three or more could alternatively be used. Similarly with the protrusions 652 and the rib 654, different numbers of each feature could be provided.

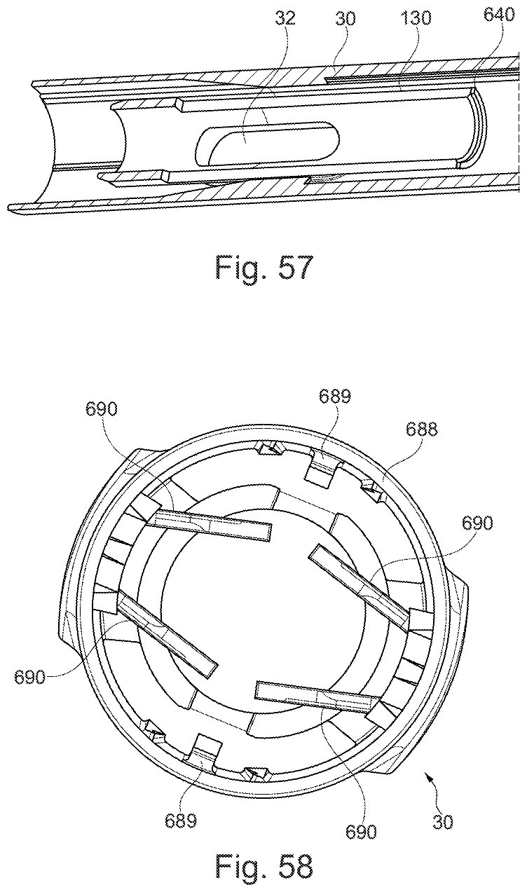

FIG. 57 shows another example of a damping portion. In this case the damping portion is a ring 640 attached to the syringe carrier 130, and the syringe carrier 130 is an integral part of the housing 30.

In general, the damping portion is flexible so that it can change in width in the longitudinal direction when compressed. As outlined above, this can be provided by a flexible material and/or by a flexible structure. The material of the damping portion could be the same as the material for the rest of the syringe carrier, or could be a different material from the rest of the syringe carrier. One option for the material of the damping portion is a thermoplastic elastomer (TPE), although other flexible materials could also be used. Another possibility is to vary material properties across the damping portion to increase the force required to squash beyond a certain point. This provides damping whilst minimising the variability of needle depth if the force of injection varies during injection. With a spring providing the force, for example, the force can decrease during injection as the spring length increases. This could also be done with a damping portion similar to the damping portion in FIG. 27, by providing a lattice structure in which initial compression of the damping portion is relatively easy and further compression of the damping portion requires greater force.

In general, the damping portion may provided in various different ways—for example, the damping portion could be overmoulded on to the syringe carrier or could be a separate component that is attached to the distal end of the syringe carrier (or placed adjacent to the distal end of the syringe carrier) during assembly. The damping portion is preferably symmetrical, particularly when it is a separate component, so that it has multiple correct orientations during assembly—this may simplify assembly. Two or more damping portions could also be combined—for example a TPE ring plus a spring.

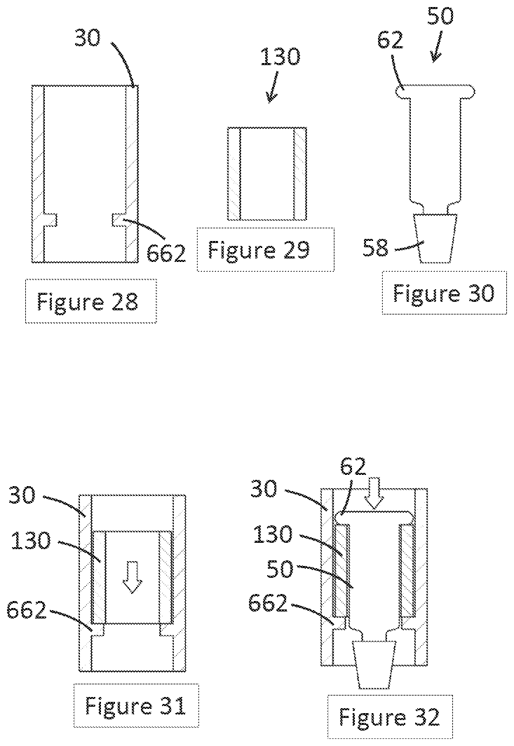

In an example as depicted in FIGS. 28 to 32, the syringe carrier 130 is tubular (more specifically cylindrical) in shape (FIG. 29). A housing 30 (FIG. 28) is also tubular, and a rib 662 (or protrusions) extends towards the axis from an inner surface of the housing 30. As shown in FIG. 31, the syringe carrier 130 is inserted into the housing from the distal end of the housing. The syringe is then inserted into the syringe carrier and the housing (FIG. 32)(though the syringe could also be inserted into the syringe carrier before being inserted into the housing).

Axial movement of the syringe in either direction (distal or proximal) can also be restricted by the syringe carrier. This can be provided by various other clips or fasteners. This design could also be used in combination with some of the other syringe carrier designs described herein, for example the design in FIGS. 1 to 5 or the design in FIGS. 6 to 10, with the housing supporting the syringe carrier.

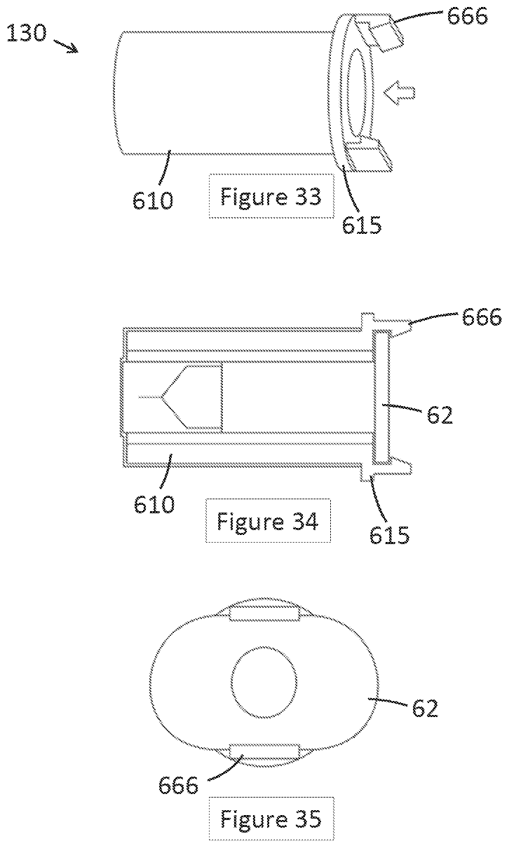

Another example (FIGS. 33 to 35) provides for axial movement of the syringe in either direction (distal or proximal) to be restricted by the syringe carrier. In this example, a snap-fit fixture 666 (in this case a distally extending protrusion with an inwardly extending protrusion on the distal end of the distally extending protrusion) at the distal end of the syringe carrier 130 engages the flange of the syringe. FIG. 33 shows the syringe carrier, FIG. 34 shows the syringe inserted in the syringe carrier, and FIG. 35 shows a view in the axial direction 13 of the syringe inserted in the syringe carrier.

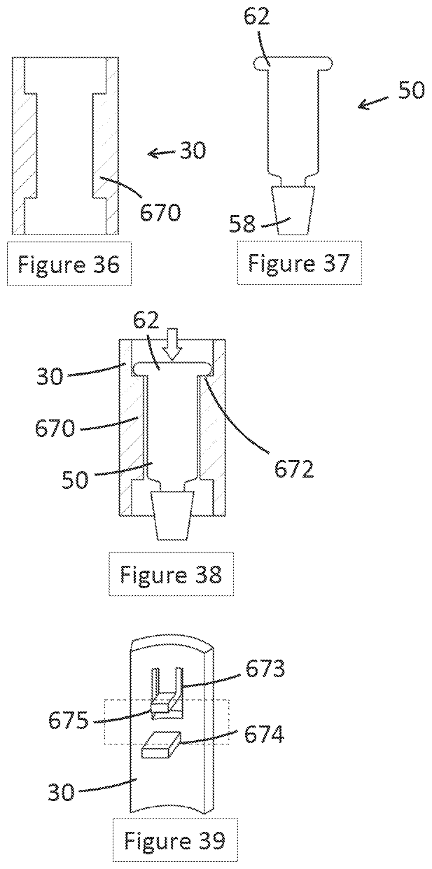

In another example, the syringe carrier is no longer a separate component, and the functionality of the syringe carrier is instead provided by a housing 30 (or body)(FIGS. 36 to 39). The housing (FIG. 36) comprises one or more tubular sections 670 that have an inner diameter that is the same as (or slightly larger than) the outer diameter of the syringe (one tubular section in the example shown in FIG. 37). The syringe is shown in the housing in FIG. 38. A distal surface 672 of the tubular section 670 supports the flange 62 of the syringe 50. As shown in FIG. 39, the housing can comprise a flexible arm. When a flexible arm is provided: when the flange of the syringe passes the arm of the housing, the arm will be bent outwards (pushed away from the axis). Subsequently, when the flange of the syringe is in final position, the arm will rebound (back towards the axis) and the flange of the syringe abuts a distal end of the tubular section of the housing (which has a smaller inner diameter than the section of the housing that is distal from the tubular section). The arm 673 (or in this case a protrusion 675 extending from the arm 673) then restricts movement of the syringe in the distal direction relative to the housing. Alternatively, the flange of the syringe can abut a protrusion 674 of the housing 30 as shown in FIG. 39, rather than abutting a distal surface 672 of the tubular section 670 as shown in FIG. 38.

The arm can restrict movement of the syringe in the distal direction relative to the housing. When an arm is not provided, another feature of the housing or of another medicament delivery device component can be used to restrict movement of the syringe in the distal direction relative to the housing.

In another example, the functions of restricting proximal motion of the syringe and supporting the sides of the syringe are shifted to the housing so that there is no need to use an extra component (a syringe carrier) for this purpose. The syringe carrier effectively becomes an integral part of the housing.

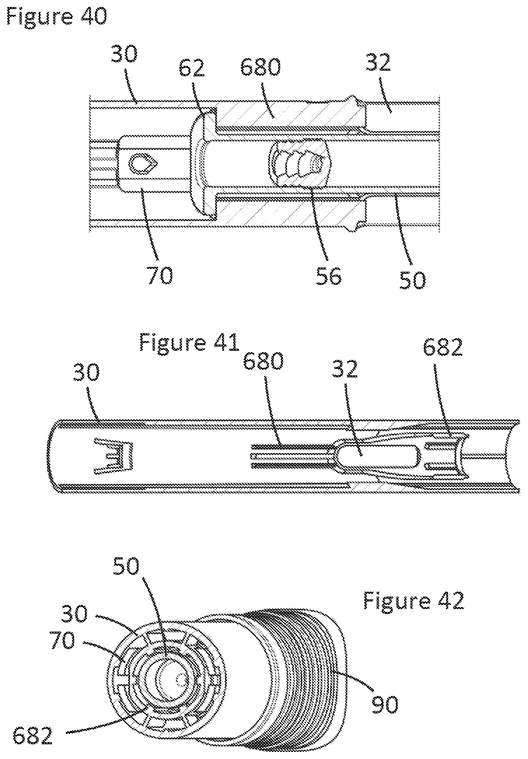

One example solution is provided in FIGS. 40 to 42. There are extra ribs 680 (distal ribs)(or equivalent structures) protruding from the inner surface of the housing 30. The radial inner gap between the extra ribs 680 is larger than the radius of the RNS and of the syringe barrel, so the extra ribs 680 will not block the assembly path of the syringe. The gap between the ribs 680 is however narrower than the radial extent of the flange 62 of the syringe 50, so the ribs 680 provide support to the flange 62 of the syringe 50 to define the most-front position of the syringe 50 after assembly. This design can remove a component and can therefore decrease complexity of assembly. A damping portion such as those described above with reference to FIGS. 16 to 27 could also be added to support the flange.

The housing 30 also comprises another set of radially inwardly protruding ribs 682 (proximal ribs) proximal to the extra ribs 680 (in this example, the extra ribs 680 are closer to the distal end of the housing than a window 32, and the radially inwardly protruding ribs 682 are further from the distal end of the housing 30 than a window 32). As with the extra ribs 680, the radially inwardly protruding ribs 682 can support the side of the syringe 50 for better stability. There can additionally or alternatively be flexible materials or structures such as a TPE or rubber pad applied on an area of the inner surface of the housing, which can thereby contact the flange of the syringe to provide flexible support.

Although two separate sets of ribs are described above, the functionality of the two sets of ribs could be combined in a single set of ribs.

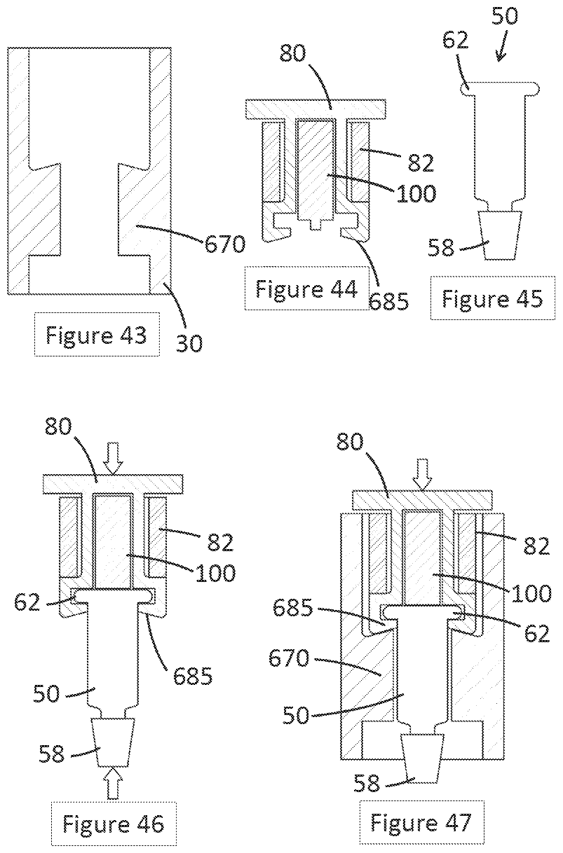

In another example, the functionality of a syringe carrier is provided by a distal housing 80 (rear housing/rear cap)(FIGS. 43 to 47). An example of a corresponding housing 30 is shown in FIG. 43; this housing shape is similar to that described above (FIG. 36) but with the distal end of the tubular section 670 describing a frusto-conical shape rather than a planar ring shape (though a planar ring shape could also be used depending on the shape of the distal housing; the advantage of a frusto-conical shape in this case is to match the shape of the proximal end of the hooks of the distal housing). The distal housing comprises an attachment point to grip the flange of the syringe, which could be a snap-fit, for example. In the example shown in FIG. 44, hooks 685 on the distal housing (in particular, on the proximal end of the distal housing) provide a snap-fit feature. Other typical medicament delivery device features, in this case a plunger rod 100 and a rotator 82, are shown for context. During assembly, the syringe is first attached to the distal housing, either by being inserted from the side (circumventing the hooks 685) or in the longitudinal direction (pushing the hooks 685 away from the axis to pass the hooks 685). The distal housing 80 and syringe 50 are subsequently inserted into the housing (FIG. 47). After insertion, movement of the hooks 685 away from the axis is restricted by the housing. In this example, both proximal and distal movement of the syringe 50 is limited by the distal housing. The distal housing 80 could be a single integral component or two or more distinct components that are subsequently attached together, for example by glue or by a snap-fit; this could simplify construction of the individual components.

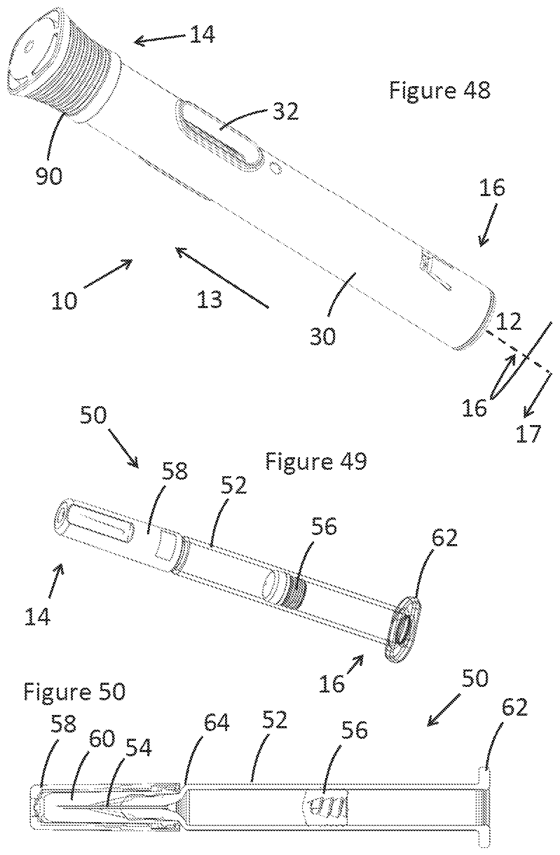

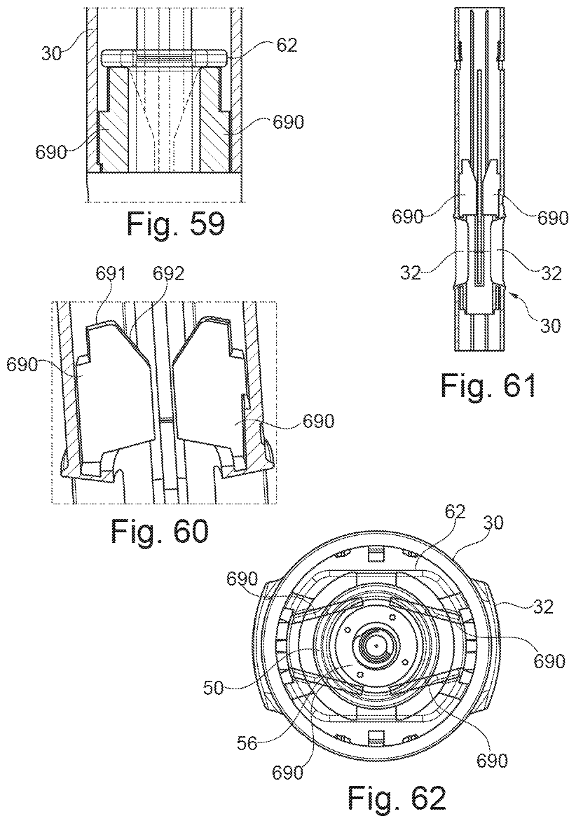

Another example is shown in FIGS. 58 to 62. In this example, the housing 30 comprises four flexible ribs 690 that extend inwards from a tubular body 688 of the housing (this tubular part of the housing is typically the outer housing, i.e. the part of the housing that can be seen in e.g. FIG. 48). The flexible ribs 690 extend inwards towards the longitudinal axis from the tubular body 688. The flexible ribs 690 extend from the tubular body 688 at an angle relative to a radial direction (that is, rather than the ribs pointing directly inwards from the tubular body towards the central axis, the ribs point in a direction that is angled relative to the radial direction). Preferably, the angle is between 10 and 90 degrees relative to the radial direction, preferably between 20 and 60 degrees, most preferably between 30 and 45 degrees. When a syringe is placed inside the tubular body, the ribs flex away from the axis and hold the syringe. In the end view of the housing in FIG. 58, the ribs 690 and the tubular body 688 of the housing can be seen, along with various other optional features such as a pair of ribs 689 that can be provided to help hold a needle cover in place relative to the housing. In FIG. 59, a cross-sectional side view can be seen, in which it can be seen that a distal surface 691 of the ribs 690 can be used to support the flange 62 of a syringe, though this is optional and another feature could provide axial support for the syringe (for example a distal surface of another component or of another part of the housing could support the flange and/or the shoulder of a syringe).

FIG. 60 shows another view of the ribs. The particular shape of the ribs is not essential, and could be varied, based for example on the shape of other medicament delivery device components such as the shape of the syringe. For example, an optional inwardly angled flexible surface 692 of the rib 690 could be provided; this can help guide the syringe during assembly. The inwardly angled flexible surface 692 of the rib 690 extends from a distal end to a proximal end, with the proximal end of the inwardly angled flexible surface 692 being closer to the axis than the distal end of the inwardly angled flexible surface 692. This design effectively results in the distal end of the ribs being narrower than the proximal end of the ribs. This design allows the syringe to be held both axially and radially by the flexible ribs. The flexible ribs could be used in addition to or instead of a damper as described elsewhere in this application.

FIG. 61 shows a cross-section side view of the housing for context, including optional windows 32 and the ribs 690. FIG. 62 shows how the syringe would fit in the housing (with the ribs 690 shown in their original pre-assembly position, rather than in the position that they would take after the ribs are flexed outwards by the syringe during assembly).

Four flexible ribs are provided in the example given with reference to FIGS. 58 to 62, but alternatively one or more ribs could be provided, with further support for the syringe optionally given by other parts of the housing or of the medicament delivery device, for example by a non-flexible part of the housing. The particular design depicted in FIGS. 58 to 62 can be beneficial, however, as the housing can provide symmetrical support for the syringe. The four ribs shown in in the example given with reference to FIGS. 58 to 62 are provided in two pairs, with each pair effectively forming a V-shape that can hold one side of a syringe, although as mentioned above, this particular angle is not essential.

In the example given with reference to FIGS. 58 to 62, the tubular housing is cylindrical with a circular cross-section, but the shape could be varied, for example with the cross-section being triangular, square or another shape. The flexible ribs 690 extend in a straight line when viewed in the direction of the axis (as shown in e.g. FIG. 58), but they could also be slightly curved rather than completely straight.

The examples herein focus on syringe carriers for autoinjectors, but the examples described herein could be implemented in other medicament delivery devices more generally, such as in pen injectors. Some of the examples herein focus on 1 ml syringes, but the designs described herein could also be used on other volumes and other types of medicament container, for example a syringe without an attached needle rather than a syringe with an attached needle. An example of an autoinjector 10 that could comprise the syringe carriers described herein is shown in FIG. 48. The example autoinjector extends along a longitudinal axis 12 in an axial direction 13 between a distal end 15 and a proximal end 14, with a radial direction 17 and a circumferential direction 16 also depicted for reference. A housing (or body) 30 and a cap 90 of the autoinjector can be seen, along with an optional window 32 in the body. The autoinjector can house a syringe. The autoinjector typically includes features such as a powerpack and a needle guard (medicament delivery member guard) inside the housing. An example needle guard 70 is shown in FIGS. 11 to 15, and in this example comprises a ring at a proximal end and two arms extending from a distal end of the ring, with an inner surface of the arms interacting with the flexible arms of the syringe carrier. However, this particular shape is not essential, and the inner surface that interacts with the flexible arms of the syringe carrier could be on the ring instead, for example. Optionally, the distal end of the needle guard is configured to activate the device, for example by rotating a rotator, but other activation mechanisms could instead be provided, such as a button.

The shape of the housing and of the cap could be varied from those shown in the example—for example, the housing could be triangular in cross section perpendicular to the axis rather than circular, could be an irregularly-shaped tube rather than a cylinder, and/or the housing could be two or more components rather than a single component. The autoinjector shown does not have an activation button, though one could be provided (i.e. a three-step autoinjector rather than a two-step autoinjector).

FIGS. 49 and 50 show an example of a syringe for reference. This particular syringe 50 comprises a medicament holder (medicament container) 52, a needle 54, a stopper 56, a rigid needle shield (RNS) 58, a flexible needle shield (FNS) 60, a flange 62 and a shoulder 64. The syringe extends from a proximal end 14 to a distal end 15. The medicament holder 52 is tubular (specifically cylindrical in this example), with the flange 62 at the distal end of the medicament holder 52 and the needle 54 at the proximal end of the medicament holder 52. The stopper 56 is in the medicament holder 52. The flexible needle shield 60 extends around the needle 54, and the rigid needle shield 58 extends around the flexible needle shield. The shoulder 64 is the proximal end of the medicament holder 52. One particular example syringe is described here, but other syringes could be used. For example, a needle 54 is included in examples described herein, but other medicament delivery members such as jet injectors could alternatively be used, or the needle could be provided separately rather than as an integral part of the syringe. A needle shield comprising a rigid needle shield 58 and a flexible needle shield 60 is included in examples described herein, but the examples described herein could be used with needle shields without a flexible needle shield or even entirely without a needle shield, although the examples described herein can be particularly beneficial when used with syringes with an RNS. The syringe could be various sizes, including but not limited to 1 ml and 2.25 ml.

Example mechanical powerpacks are described herein (for example part of a powerpack is shown in FIG. 44), but other types of powerpack could be used instead, for example an electrically powered powerpack or a gas-powered powerpack. An example of a device in which syringe carriers as described herein could be used is provided in WO2011/123024, which is hereby incorporated by reference.

A base 132 is described herein. Typically, the base is depicted as the distal portion of the syringe carrier, but could alternatively be spaced apart in the axial direction from the distal end of the syringe carrier.

Arms 134 are described herein. Most (though not all) of the examples herein use two arms, although in the examples with two arms, one, three or more arms could alternatively be provided. Similarly, other features that are provided (arms, protrusions, cut-outs, recesses and the like) can generally be provided in a quantity different to the specific number described in the examples given.

Many of the syringe carriers described herein comprise some kind of optional protrusion or rib. For example, a number of the examples, including the syringe carrier shown in FIG. 16, comprise two ribs 138, four second ribs 139 and two protrusions 140. The ribs 138 extend in the longitudinal direction, with the proximal end of each rib 138 attached to an arm 134 and the distal end of each rib 138 attached to the base 132. The second ribs 139 extend in the longitudinal direction. A second rib 139 is arranged on each side of each arm 134 in the circumferential direction. The protrusions 140 are attached to the base 132. Another optional feature is a distal flange 148. The flange 148 is attached to the base 132 and extends in the radial direction away from the axis and extends in the circumferential direction around the base. Protrusions 646 are another example. These ribs, protrusions and flanges can provide various advantages, including helping to align the syringe carrier relative to other features during assembly, maintaining rigidity of the syringe carrier, and/or fixing the position of the syringe carrier relative to other components of a medicament delivery device in a completed device. Whilst features such as the protrusions 140, ribs 138, 139 and flanges 148 are depicted as having a particular shape, these shapes could be varied depending on factors such as the desired rigidity and on the shape of other components within a particular design of medicament delivery device.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

Various modifications to the embodiments described are possible and will occur to those skilled in the art without departing from the present disclosure which is defined by the following claims.

Some features are summarised in the clauses below.

1. A syringe carrier for a syringe with a flange, the syringe carrier comprising a tubular housing extending along a longitudinal axis from a proximal end to a distal end and a syringe holder attached to the distal end of the tubular housing, wherein the syringe holder is configured to hold the flange of the syringe, and wherein the syringe holder comprises either a c-clip or a screw thread.

2. The syringe carrier of clause 1, wherein the syringe holder comprises a c-clip, and wherein the c-clip extends more than half of the way around the circumference relative to the longitudinal axis, and preferably more than 270 degrees around the circumference relative to the longitudinal axis.

3. The syringe carrier of clause 1 or 2, wherein the syringe holder comprises a c-clip, and wherein the c-clip is flexible to allow the diameter of the c-clip to expand to allow the flange of the syringe to pass the c-clip during medicament delivery device assembly.

4. The syringe carrier of any of clauses 1 to 3, wherein the syringe holder comprises a c-clip, and wherein the c-clip comprises two inwardly extending ribs extending around the circumference of the c-clip, wherein the two inwardly extending ribs are spaced apart from one another in the direction of the longitudinal axis so that the flange of the syringe can fit between the two inwardly extending ribs.

5. The syringe carrier of clause 1, wherein the syringe holder comprises a screw thread, and wherein the screw thread extends towards the longitudinal axis from an inside surface of the syringe holder.

6. The syringe carrier of any of clauses 1 to 5, wherein the syringe holder is tubular.

7. A sub-assembly for a medicament delivery device, the sub-assembly comprising a syringe carrier according to any of clauses 1 to 6 and a syringe with a flange, wherein the syringe is inside the syringe carrier and the flange is inside the syringe holder.

8. A medicament delivery device comprising the syringe carrier of any of clauses 1 to 6 or the sub-assembly of clause 7, wherein the medicament delivery device is optionally an autoinjector.

9. A method of inserting a syringe into a syringe carrier, the method comprising the steps of:
providing a syringe carrier according to any of clauses 1 to 6 and providing a syringe with a flange; and
inserting the syringe into the syringe carrier in the direction of the longitudinal axis.

10. The method of clause 9, wherein the syringe is inserted into the distal end of the syringe carrier and moved in the proximal direction relative to the syringe carrier until the flange of the syringe engages the syringe holder.

11. The method of clause 9 or 10, wherein the syringe holder comprises a screw thread and wherein the syringe is rotated relative to the syringe carrier during insertion of the syringe into the syringe carrier so as to engage the flange of the syringe with the screw thread.

12. A medicament delivery device extending along a longitudinal axis in an axial direction from a distal end to a proximal end, the medicament delivery device comprising:
a housing;
a medicament delivery member guard attached to the housing and axially moveable relative to the housing from a proximal position to a distal position, the medicament delivery member guard comprising a surface that faces towards the longitudinal axis and extends in the axial direction;
a tubular syringe carrier attached to the housing, wherein the syringe carrier comprises a flexible arm at a proximal end of the syringe carrier, the flexible arm comprising a protrusion extending towards the longitudinal axis; and
a syringe inside the syringe carrier,
wherein, when the medicament delivery member guard is in the distal position, the surface of the medicament delivery member guard is adjacent to the flexible arm of the syringe carrier so as to restrict movement of the protrusion of the flexible arm away from the longitudinal axis,
and when the medicament delivery member guard is in the proximal position, movement of the protrusion of the flexible arm away from the longitudinal axis is not restricted by the surface of the medicament delivery member guard, so that the protrusion of the flexible arm is free to move away from the longitudinal axis.

13. The medicament delivery device of clause 12, wherein the flexible arm of the syringe carrier is attached to the rest of the syringe carrier at a distal end of the arm.

14. The medicament delivery device of clause 12 or 13, wherein the flexible arm of the syringe carrier comprises an outwardly extending protrusion, and wherein, when the medicament delivery member guard is in the distal position, the surface of the medicament delivery member guard is adjacent to the outwardly extending protrusion.

15. The medicament delivery device of any of clauses 12 to 14, wherein there is a gap between the protrusion of the flexible arm and a shoulder of the syringe carrier when the medicament delivery member guard is in the proximal position and there is no gap between the protrusion of the flexible arm and the shoulder of the syringe carrier when the medicament delivery member guard is in the distal position.

16. The medicament delivery device of any of clauses 12 to 15, comprising a damper at a distal end of the syringe carrier.

17. The medicament delivery device of clause 16, wherein the damper is an integral part of the syringe carrier.

18. The medicament delivery device of clause 16, wherein the damper is a separate component at the distal end of the syringe carrier

19. The medicament delivery device of any of clauses 16 to 18, wherein the damper is a compression spring.

20. The medicament delivery device of any of clauses 16 to 18, wherein the damper is a flexible ring or a flexible protrusion.

21. The medicament delivery device of any of clauses 16 to 20, wherein the damper is made of a flexible material such as a thermoplastic elastomer.

22. The medicament delivery device of any of clauses 12 to 21, wherein the medicament delivery member guard comprises a ring at a proximal end and two arms extending from a distal end of the ring.

23. The medicament delivery device of clause 22, wherein the surface of the medicament delivery member guard is an inner surface of one of the arms of the medicament delivery member guard.

24. The medicament delivery device of any of clauses 12 to 23, wherein the tubular syringe carrier is an integral part of the housing.

25. The medicament delivery device of any of clauses 12 to 24, wherein the medicament delivery device is an autoinjector.

26. A housing of a medicament delivery device, wherein the housing is tubular and comprises a syringe carrier.

27. The housing of the previous clause, comprising a rib extending inwardly from an inner side of the housing, the rib comprising a distally facing surface arranged to support a flange of a syringe.

28. The housing of either of the previous two clauses, comprising a second rib extending inwardly from an inner side of the housing, the rib comprising an inwardly facing surface arranged to support a side of a syringe.

29. The housing of any of the previous three clauses, wherein the housing is a distal housing.

30. A syringe carrier, wherein the syringe carrier comprises a damping portion at the distal end.

31. The syringe carrier of clause 30, wherein the damping portion is made of a flexible plastic such as a thermoplastic elastomer.

32. The syringe carrier of clause 30 or 31, wherein the damping portion is a spring.

33. The syringe carrier of clause 30 or 31, wherein the damping portion comprises two separate portions arranged opposite one another relative to the axis.

34. A method of manufacturing a syringe carrier, the method comprising the steps of: moulding a syringe carrier; and overmoulding a damping portion on the distal end of the syringe carrier.

35. A medicament delivery device comprising the syringe carrier of any of clauses 30 to 33 or comprising the housing of any of clauses 26 to 29.

36 The previous clause, in which the medicament delivery device is an autoinjector.

37. A method of assembling a medicament delivery device, comprising the steps of attaching a syringe to a syringe carrier and of subsequently inserting the syringe and syringe carrier into a housing.

38. A method of assembling a medicament delivery device, comprising the steps of attaching a syringe carrier to a housing and of subsequently inserting a syringe into the syringe carrier and the housing.

39. A damping ring for a medicament delivery device, the damping ring being made of a flexible material, the damping ring comprising two arms, wherein the arms are configured to engage a component of said medicament delivery device to hold the damping ring in place relative to said component of said medicament delivery device.

40. A housing for a medicament delivery device, the housing extending along a longitudinal axis from a proximal end to a distal end, the housing comprising a tubular body and one or more flexible ribs extending inwards towards the longitudinal axis from the tubular body, wherein the flexible ribs extend from the tubular body at an angle relative to a radial direction relative to the axis so that, when a syringe is placed inside the tubular body, the one or more flexible ribs flex away from the axis and hold the syringe.

41. The housing of the previous clause, wherein the angle is between 10 and 90 degrees, preferably between 20 and 60 degrees, most preferably between 30 and 45 degrees.

42. The housing of one of the previous two clauses, wherein four flexible ribs are provided.

43. A medicament delivery device comprising the damper ring of clause 39 or the housing of any of clauses 40 to 42.

44. The previous clause, in which the medicament delivery device is an autoinjector.

45. A method of assembling a medicament delivery device, comprising inserting a syringe into a housing so that the syringe causes a rib inside a tubular body of the housing to flex away from a longitudinal axis of the housing, thereby supporting the syringe within the housing.

1-15. (canceled)

16. A syringe carrier for a syringe with a flange, the syringe carrier comprising a tubular housing extending along a longitudinal axis from a proximal end to a distal end and a syringe holder attached to the distal end of the tubular housing,
wherein the syringe holder is configured to hold the flange of the syringe, and
wherein the syringe holder comprises either a c-clip or a screw thread.

17. The syringe carrier of claim 16, wherein the syringe holder comprises a c-clip, and wherein the c-clip extends more than half of the way around the circumference of relative to the longitudinal axis, and preferably more than 270 degrees around the circumference of relative to the longitudinal axis.

18. The syringe carrier of claim 16, wherein the syringe holder comprises a c-clip, and wherein the c-clip is flexible to allow the diameter of the c-clip to expand to allow the flange of the syringe to pass the c-clip during medicament delivery device assembly.

19. The syringe carrier of claim 16, wherein the syringe holder comprises a c-clip, and wherein the c-clip comprises two inwardly extending ribs extending around the circumference of the c-clip, wherein the two inwardly extending ribs are spaced apart from one another in the direction of the longitudinal axis so that the flange of the syringe can fit between the two inwardly extending ribs.

20. The syringe carrier of claim 16, wherein the syringe holder comprises a screw thread, and wherein the screw thread extends towards the longitudinal axis from an inside surface of the syringe holder.

21. A medicament delivery device comprising the syringe carrier of any of claim 16.

22. A method of inserting a syringe into a syringe carrier, the method comprising the steps of:
providing a syringe carrier according to claim 16 and providing a syringe with a flange; and
inserting the syringe into the syringe carrier in the direction of the longitudinal axis.

23. The method of claim 22, wherein the syringe is inserted into the distal end of the syringe carrier and moved in the proximal direction relative to the syringe carrier until the flange of the syringe engages the syringe holder.

24. A medicament delivery device extending along a longitudinal axis in an axial direction from a distal end to a proximal end, the medicament delivery device comprising:
a housing;
a medicament delivery member guard attached to the housing and axially moveable relative to the housing from a proximal position to a distal position, the medicament delivery member guard comprising a surface that faces towards the longitudinal axis and extends in the axial direction;
a tubular syringe carrier attached to the housing, wherein the syringe carrier comprises
a flexible arm at a proximal end of the syringe carrier, the flexible arm comprising a protrusion extending towards the longitudinal axis; and
a syringe inside the syringe carrier,
wherein, when the medicament delivery member guard is in the distal position, the surface of the medicament delivery member guard is adjacent to the flexible arm of the syringe carrier so as to restrict movement of the protrusion of the flexible arm away from the longitudinal axis,
and when the medicament delivery member guard is in the proximal position, movement of the protrusion of the flexible arm away from the longitudinal axis is not restricted by the surface of the medicament delivery member guard, so that the protrusion of the flexible arm is free to move away from the longitudinal axis.

25. The medicament delivery device of claim 24, wherein the flexible arm of the syringe carrier is attached to the rest of the syringe carrier at a distal end of the arm.

26. The medicament delivery device of claim 24, wherein the flexible arm of the syringe carrier comprises an outwardly extending protrusion, and wherein, when the medicament delivery member guard is in the distal position, the surface of the medicament delivery member guard is adjacent to the outwardly extending protrusion.

27. The medicament delivery device of claim 24, wherein there is a gap between the protrusion of the flexible arm and a shoulder of the syringe carrier when the medicament delivery member guard is in the proximal position and there is no gap between the protrusion of the flexible arm and the shoulder of the syringe carrier when the medicament delivery member guard is in the distal position.

28. The medicament delivery device of claim 24, comprising a damper at a distal end of the syringe carrier.

29. The medicament delivery device of claim 24, wherein the medicament delivery member guard comprises a ring at a proximal end and two arms extending from a distal end of the ring.

30. The medicament delivery device of claim 21, wherein the medicament delivery device is an autoinjector.

31. An assembly comprising:
a syringe having a flange; and
a syringe carrier for a syringe with a flange, the syringe carrier comprising:
a tubular housing extending along a longitudinal axis from a proximal end to a distal end and a syringe holder attached to the distal end of the tubular housing,
wherein the syringe holder surrounds and is engaged with the flange through a connector.

32. The assembly of claim 31, wherein the connector comprises a c-clip that extends more than half of the way around the circumference relative to the longitudinal axis and is flexible such that the c-clip expands to allow the flange of the syringe to pass through the c-clip axially during medicament delivery device assembly.

33. The assembly of claim 31, wherein the connector comprises a screw thread that extends towards the longitudinal axis from an inside surface of the syringe holder.
